(19) United States
(12) Patent Application Publication
(10) Pub. No.: US 2008/0045972 A1
(43) Pub. Date: Feb. 21, 2008

(54) ADAPTOR FOR ATTACHING A REFERENCE ARRAY TO A MEDICAL INSTRUMENT HAVING A FUNCTIONAL DIRECTION OR PLANE

(76) Inventors: Benjamin Wagner, Munich (DE); Timo Neubauer, Poing (DE); Mario Schubert, Poing (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(21) Appl. No.: 11/840,526

(22) Filed: Aug. 17, 2007

Related U.S. Application Data
(60) Provisional application No. 60/823,846, filed on Aug. 29, 2006.

(30) Foreign Application Priority Data
Aug. 18, 2006 (EP) 06017265

(51) Int. Cl. A61B 19/00

(52) U.S. Cl. 606/130

(57) ABSTRACT

An adaptor for attaching a reference array to a medical instrument, said instrument having a functional direction or plane includes a reference array mount for attaching the reference array to the adapter, an instrument engagement section for attaching the adaptor to the instrument, and a self-aligning coupling mechanism. The coupling mechanism is coupled to the reference array mount and to the instrument engagement section and is operative to move the reference array attached to the reference array mount to a predetermined location relative to the functional direction or plane of the instrument when the instrument engagement section is attached to the instrument.

### RELATED APPLICATION DATA

This application claims priority of U.S. Provisional Application No. 60/823,846 filed on Aug. 29, 2006, which is incorporated herein by reference in its entirety.

### FIELD OF THE INVENTION

The invention relates to medical navigation and, more particularly, to an adaptor for attaching a reference array to a medical instrument having at least one functional plane.

### BACKGROUND OF THE INVENTION

Reference arrays serve to make an instrument visible to and trackable by a medical tracking and navigation system. It is also possible to identify each individual reference array (e.g., based on a specific arrangement of markers on the reference array) and, therefore, identify each individual instrument attached to the reference array.

In some cases, interfaces configured in a particular way may be provided on instruments, and reference arrays are then fastened thereto using an adaptor. These adaptors can comprise a mounting for the reference array and an instrument engagement, wherein the instrument engagement is specifically configured such that it can cooperate with the interface on the instrument.

To perform navigation, the instruments can be assigned to specific reference arrays, and this assignment can be stored within memory of a navigation system (e.g., the shape or profile of the reference array can be stored in memory of the navigation system). The navigation system then can identify the instrument on the basis of the reference array. Moreover, the navigation system can determine where particular functional regions of the instrument (e.g., the instrument tip) are situated at any time.

One disadvantage of this methodology is that either a large database of pre-calibrated instruments and reference arrays need be provided and maintained, or each instrument that is provided with a reference adaptor has to be assigned to the reference adaptor, and the shape of the instrument has to be identified or calibrated (e.g., before use of the instrument the location of the instrument's functional portion relative to the reference adapter is identified). Further, the locations on the instrument in which a reference adapter may be attached may be limited.

### SUMMARY OF THE INVENTION

An adaptor includes an instrument engagement section that is couplable to an instrument. The instrument engagement section can be a clamping engagement that may be adjusted in size and/or (to a certain extent) shape. The adapter also includes a reference array mount coupled to the instrument engagement section via a self-aligning coupling mechanism. When the adaptor is attached to the instrument, the coupling mechanism moves the reference array, which is coupled to the reference array mount, into a predetermined location relative to a functional direction or plane of the instrument.

In other words, the adapter can be based on the realization that for some instruments, it is not absolutely necessary to completely know the location and position of the functional parts of the instrument. Rather, in some cases it is perfectly sufficient if the reference array only provides an alignment of a particular direction or plane as information for the navigation system. Such a functional direction or plane of the instrument, for example, can be a direction or plane that determines or identifies a working direction of the instrument. Thus, if an instrument, for example, is to be advanced or retracted in a particular direction, it is often sufficient to confirm that this particular direction is maintained. If, during application of the instrument, the adaptor moves the reference array to a predetermined location with respect to such a functional direction or plane of the instrument, it is possible to establish by navigation whether the plane or direction has been maintained during the application. An advantage of this is that navigation can be performed without storing the dimensions of the instrument in a database. This enables navigation of instruments without exchanging or otherwise providing the instrument technical data to the navigation system. Thus, the adaptor forms a sort of universal adaptor for a large number of instruments from various manufacturers.

The instrument, for example, can be a bone broach, and a functional plane of the instrument can be a longitudinal mid-plane of the broach, in particular the broach handle. In this embodiment, the instrument provides a universal adaptor for reference arrays that may be fastened to the instrument, such as broaches. An anteversion angle can therefore be navigated and verified while using the broach to broach a femur, for example, when performing a hip replacement operation. Such a universal broach adaptor makes it possible to navigate and verify the anteversion angle of broaches whose dimensions do not have to be known as a dataset or ascertained by calibration processes. Since the adaptor allows the reference array to be arranged at a predetermined location on the handle of the broach, the longitudinal mid-plane of the broach is known after the reference array has been attached, and the navigation-assisted application can begin. It is advantageous if the instrument to which the adaptor is to be fastened has engaging areas that are symmetrical with respect to the functional direction or plane of the instrument.

The instrument engagement section can include two or more engaging elements that act counter to each other, and in any position of the engaging elements, the direction of action of the instrument engagement section can have the same positional relationship to the alignment of the reference array. In other words, the direction of force for holding the adaptor on the instrument can be kept in the same relationship to the alignment of the reference array, regardless of whether the engaging elements is more or less open. The line of force, for example, always can be parallel to the alignment of the reference array.

The reference array can define a reference array plane that is perpendicular to the functional direction or plane. For any opening angle of the instrument engagement section, the coupling mechanism can place the reference array plane perpendicular to the functional direction or plane, wherein the reference array advantageously defines the location of the functional direction or plane. In other words, the navigation system can know that for a particular arrangement of markers lying on the reference array in a plane, the point of intersection with the perpendicular functional direction or plane lies at a particular point on this plane.

A coupling mechanism of the adapter can be biased by a spring pressure that generates an engaging force action on the instrument. The coupling mechanism can comprise two mutually intersecting arms that, at the point of intersection, are connected in a joint and comprise the instrument engagement section on one side of the point of intersection and the reference array mount on the other side.

The instrument engagement section can comprise two or more contact pieces, such as contact jaws, which can be applied to opposite sides of the instrument and can be mounted on the coupling mechanism in a joint, in particular at two opposite ends of the arms. With regard to the reference array mount, this can comprise a holding element that may be directly connected to the reference array and elastically clamped (e.g., a spring clamp) such that it can pivot in a joint in the coupling mechanism (e.g., between opposite ends of the arms). In such a configuration, the holding element, for example, can be clamped between two tension springs arranged in a joint on the arm ends, wherein the tensions springs generate the engaging force action on the instrument engagement section at the other end of the arms. Another possible configuration is to attach the holding element in a joint at the point of intersection of the arms on the side facing away from the reference array.

### BRIEF DESCRIPTION OF THE DRAWINGS

The forgoing and other features of the invention are hereinafter discussed with reference to the drawing.

FIG. 1 illustrates an exemplary adaptor in accordance with the invention, wherein a reference array is attached to the adapter.

FIG. 2 illustrates the adaptor and reference array of FIG. 1 attached to a bone broach.

FIG. 3 is a schematic view of an exemplary adaptor, reference array and broach from a front-facing side.

FIG. 4 is a side view of a front-facing side of the adapter, reference array and instrument, wherein the adaptor is attached to the instrument via oblique outer areas.

FIG. 5 illustrates a bone broach including an exemplary adaptor in accordance with the invention, and a navigation system for providing surgical assistance.

### DETAILED DESCRIPTION

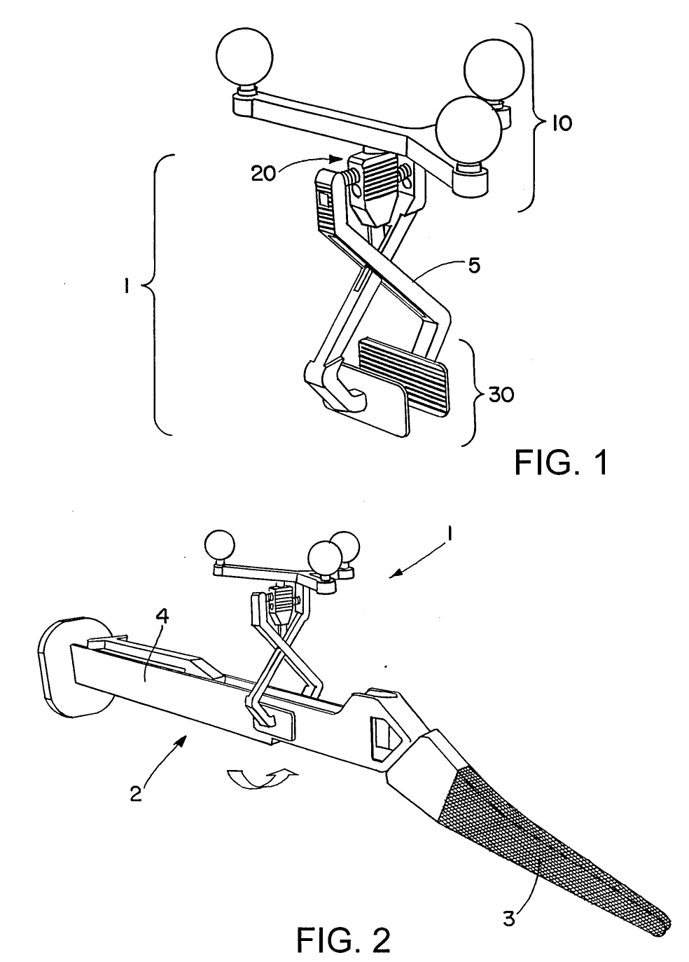

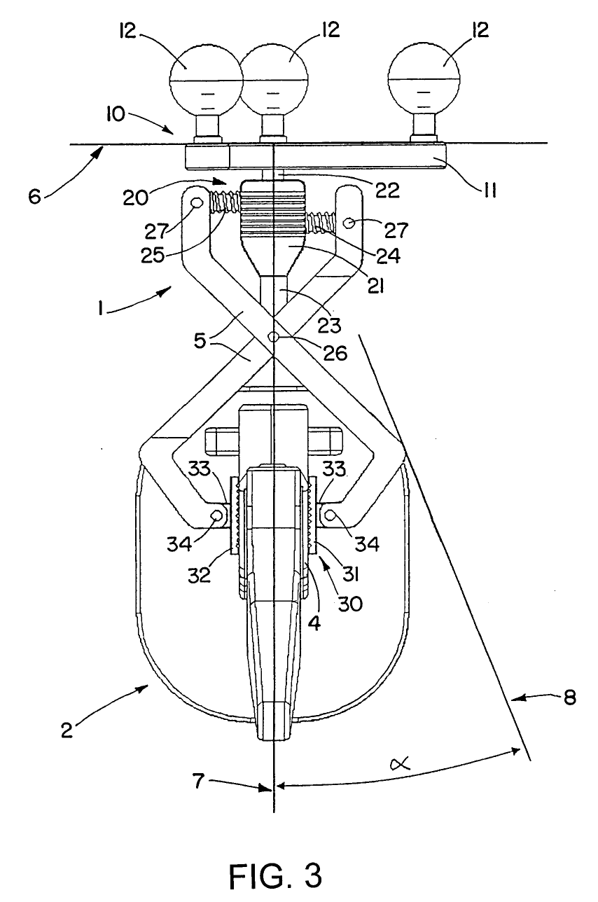

FIGS. 1 and 2 show a perspective view of the main components of an exemplary adaptor attaching system for reference arrays, while FIG. 3 provides a schematic view of the adapter attaching system. The adaptor 1 can broadly comprise a reference array mount 20 for mounting a reference array 10 to the adapter 1, an instrument engagement section 30 for coupling the adapter 1 to an instrument, and a coupling mechanism 5 coupling the reference array mount 20 to the instrument engagement section 30. The adapter 1 also may include the reference array 10, which can comprise a plurality of markers 12 mounted on a star-like marker support 11, for example. The adaptor 1, via the instrument engagement section 30, can be clamped to the instrument together with the reference array 10, and is shown in this state in FIG. 2 on a bone broach 2 having a handle 4 and a broaching portion 3.

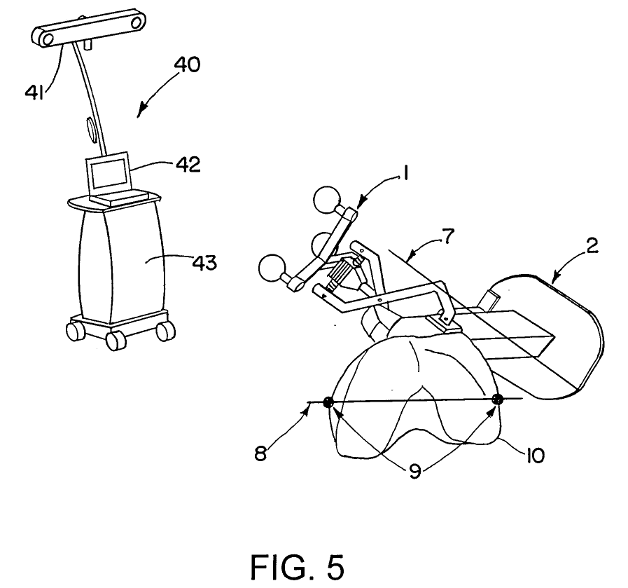

In the state shown in FIG. 2, the broach 2 can be navigated, for example, in a navigation environment such as is shown in FIG. 5 using a navigation system 40 (which can include a data processing unit 43 and screen output 42). A tracking system 41, such as a camera-based tracking system 41, can be assigned to the navigation system 40. Further discussion with respect to FIG. 5 is provided below.

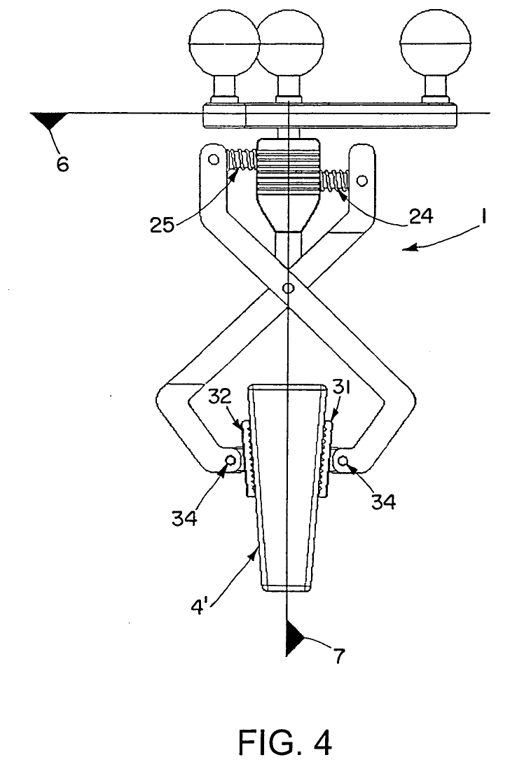

FIG. 3 illustrates the particulars of an attaching system comprising the instrument 2 (e.g., broach), the adaptor 1 and the reference array 10. The view of the front-facing side shows that the handle 4 of the broach 2 comprises outer walls or sides (e.g., parallel walls), which the instrument engagement section 30 engages on both sides. A longitudinal mid-plane of the broach 2 is indicated by the reference sign 7 and illustrated as a line in this view. On its outer sides, the handle 4 of the broach 2 runs parallel to and is symmetrically about the longitudinal mid-plane 7. Engaging elements 31 and 32, which, for example, can be provided with friction-enhanced surfaces (in this case, corrugated surfaces), engage on the sides of the handle 4 in order to ensure sufficient grip. It is also possible, for example, to use rubber-like inserts. The engaging elements 31 and 32 can have projecting extensions 33 that are in turn attached to the arms 5 of the adaptor 1 such that they can pivot at joint 34. The ability to pivot will be discussed again below with reference to FIG. 4. The arms 5 of the adaptor 1 can intersect (e.g., scissor arms) and can be connected to each other via joint 26. Another linking point 27 is situated at each end of the arms 5. Tension springs 24 and 25 can extend from the linking points 27 and can be held on a guide and terminate at the holding element 21.

The springs 24 and 25 press the holding element 21, which is in turn fastened to the joint 26 via extension 23, into a position between the upper arm parts. At this position, for any opening angle of the arms 5 in the region of the instrument engagement section 30, the plane 6 of the reference array 10 runs perpendicular to the longitudinal mid-plane 7. The reference array 10 can be attached to the holding element 21 via an extension 22, and its markers 12 can be placed on the marker support 11. In the present case, the upper side of the marker support 11 defines a profile of the plane 6, i.e., the reference array plane.

In other words, the coupling mechanism (including the instrument engagement section 30, the arms 5 and the holding element 21), together with their jointed connections and springs 24 and 25, as its individual parts, ensures that the reference array plane 6 is always perpendicular on the longitudinal mid-plane 7 of the broach 2, regardless of how wide the engaging elements 31 and 32 have been opened.

FIG. 3 also shows the anteversion angle α, which is the angle between the longitudinal mid-plane 7 and the epicondylar axis of the femur. Reference may again be made here to FIG. 5, which indicates how the epicondylar axis 8 can be ascertained. FIG. 5 shows the end of the femoral bone 10, and the epicondylar axis 8 can be determined by the two epicondylar points 9. While the bone 10 is broached using the broach 2, the longitudinal mid-plane 7 should be in a predetermined relationship to the epicondylar axis 8. The longitudinal plane 7 specifically can be navigated at any time by means of the adaptor 1, without the dimensions of the broach 2 being exactly known.

FIG. 4 shows how the adaptor 1 an adapt to instruments that do not have walls or side areas running in a straight line and/or parallel to the longitudinal mid-axis 7. FIG. 4 shows (again in a front-facing view) an instrument comprising a handle 4′ having an outer contour that narrows in a downward direction but is still symmetrical with respect to the longitudinal mid-plane 7. Due to the jointed 34 provided for the engaging elements 31 and 32, the adaptor 1 adapts to the profile of the handle surface of the instrument. Thus, a mechanism can be integrated that causes the two engaging elements 31 and 32 (e.g., clamping jaws) to move symmetrically. The result is that the longitudinal mid-plane 7 of the handle is always centered, and since the reference array plane 6 and the longitudinal mid-plane 7 are at right angles to each other, the navigation system 40 knows the longitudinal mid-plane 7 of the instrument when it detects the reference array 10, the characteristics of which can be stored in a corresponding database. For this reason, the anteversion angle can always be navigated.

One possible workflow for using a broach 2 when broaching a femur is as follows:

The femur is first prepared, and the adaptor 1 is then placed onto the broach handle 4. It is not necessary to calibrate the device as a whole, since in its configuration, the adaptor 1 is known to the navigation system 40, to which a tracking system 41 is assigned. Due to the configuration of the coupling mechanism 5, and since the engaging elements 31 and 32 (e.g., clamping jaws) and the reference array mount are arranged in a joint, the adaptor 1 aligns itself to the mid-plane of the broach handle 4, which is then perpendicular to the reference array plane 6. Since the geometry and dimensions of the reference array 10 are known to the navigation system 40, it is possible to simply determine the location of the longitudinal mid-plane 7 with respect to the reference array plane.

In the next step, the broach 2, together with its handle 4, can be aligned to the planned anteversion angle, wherein the surgeon takes the device as a whole and moves it to the proximal end of the femur, which has already been prepared for broaching. Since it is possible to navigate the planes, the surgeon can easily align the instrument to the planned anteversion angle. Aligning the planes is sufficient in this case; it is not necessary to navigate the instrument as a whole in terms of its geometry and dimensions.

The adaptor 1 then can be removed to perform the broaching process. When the adaptor 1 is removed, it no longer gets in the way of the broaching work. Moreover, the clamping adaptor can be very easily removed and re-attached to re-verify the working angle, and this specifically shows another advantage of the adaptor configuration.

Removing the adaptor is advantageous for the actual broaching work, since very large forces can occur. Verification and further work with the adaptor removed can be repeated until broaching is complete and the correct, planned anteversion angle has been achieved.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. An adaptor for attaching a reference array to a medical instrument, said instrument having a functional direction or plane, comprising:
a reference array mount for attaching the reference array to the adapter;
an instrument engagement section for attaching the adaptor to the instrument; and
a self-aligning coupling mechanism coupled to the reference array mount and to the instrument engagement section, wherein the self-aligning coupling mechanism is operative to move the reference array attached to the reference array mount to a predetermined location relative to the functional direction or plane of the instrument when the instrument engagement section is attached to the instrument.

2. The adapter according to claim 1, wherein the instrument engagement section comprises an adjustable clamping mechanism that can be adjusted in size and/or in shape.

3. The adaptor according to claim 1, wherein the functional direction or plane of the instrument is a direction or plane that determines a working direction of the instrument.

4. The adaptor according to claim 1, wherein the instrument is a bone broach, and the functional plane is a longitudinal mid-plane of the broach.

5. The adapter according to claim 4, wherein the longitudinal mid-plane of the broach is a longitudinal mid-plane of a handle of the broach.

6. The adaptor according to claim 1, wherein the instrument engagement section comprises two engaging elements that act counter to each other, and in any position of the engaging elements relative to the instrument, the direction of action of the instrument engagement section produces the same positional relationship to the alignment of the reference array.

7. The adaptor according to claim 1, wherein the reference array defines a reference array plane that is perpendicular to the functional direction or plane of the instrument.

8. The adaptor according to claim 7, wherein for any opening angle of the instrument engagement section, the coupling mechanism places the reference array plane perpendicular to the functional direction or plane.

9. The adaptor according to claim 8, wherein the reference array defines a location of the functional direction or plane of the instrument.

10. The adaptor according to claim 1, wherein the coupling mechanism is biased by a force that generates an engaging action on the instrument engagement section.

11. The adapter according to claim 10, wherein the force is generated by a spring.

12. The adaptor according to claim 1, wherein the coupling mechanism comprises two mutually intersecting arms connected by a joint so as to form two pairs of arms, and wherein a first pair of arms are coupled to the instrument engagement section and a second pair of arms are coupled to the reference array mount.

13. The adapter according to claim 12, wherein the joint is located at the point of intersection of the intersecting arms.

14. The adaptor according to claim 12, further comprising the reference array, wherein the reference array mount comprises a holding element connected to the reference array and to the coupling mechanism, said connection to the coupling mechanism being via the joint.

15. The adaptor according to claim 14, wherein the holding element is further coupled to a first arm of the second pair of arms via a first elastically loaded joint, and to a second arm of the second pair of arms via a second elastically loaded joint, and wherein the first and second elastically loaded joints generate an engagement force on the instrument engagement section.

16. The adaptor according to claim 14, wherein the holding element is attached in the joint on a side facing away from the reference array.

17. The adaptor according to claim 1, wherein the instrument engagement section comprises two contact pieces that can be applied to opposite sides of the instrument, said contact pieces each coupled to the coupling mechanism via a joint.

18. The adaptor according to claim 17, wherein the two contact pieces comprise contact jaws.

19. The adaptor according to claim 1, further comprising the reference array.
